(19) United States
(12) Patent Application Publication
(10) Pub. No.: US 2024/0344036 A1
(43) Pub. Date: Oct. 17, 2024

(54) METHOD OF GENERATING iPS CELL AND iPS CELL GENERATION SYSTEM

(71) Applicants: Canon Medical Systems Corporation, Otawara-shi (JP); CANON KABUSHIKI KAISHA, Tokyo (JP)

(72) Inventor: Katsuaki DEGUCHI, Otawara (JP)

(73) Assignees: Canon Medical Systems Corporation, Otawara-shi (JP); CANON KABUSHIKI KAISHA, Tokyo (JP)

(21) Appl. No.: 18/635,082

(22) Filed: Apr. 15, 2024

(30) Foreign Application Priority Data
Apr. 17, 2023 (JP) 2023-067234

(51) Int. Cl. C12N 5/074

(52) U.S. Cl. CPC C12N 5/0696; C12N 2501/599; C12N 2506/11; C12N 2506/115

(57) ABSTRACT

According to one embodiment, a method of generating an iPS cell includes extracting monocytes from mononuclear cells separated from whole blood to separate the mononuclear cells into the monocytes and non-monocytes, reprogramming the separated monocytes to generate a monocyte-derived iPS cell, and culturing the separated non-monocytes in a first culture medium to prepare first raw material cells for iPS cell generation.

### CROSS-REFERENCE TO RELATED APPLICATIONS

This application is based upon and claims the benefit of priority from Japanese Patent Application No. 2023-067234, filed Apr. 17, 2023, the entire contents of which are incorporated herein by reference.

### FIELD

Embodiments described herein relate generally to a method of generating an iPS cell and an iPS cell generation system.

### BACKGROUND

Currently, in establishing induced pluripotent stem cells (iPS cells) from blood collected from a donor, iPS cells are established from a specific cell type in blood. For example, T cells are isolated from blood and reprogrammed to establish iPS cells from T cells. However, due to recombination occurring in T cell receptor genes of T cells, T cell-derived iPS cells have DNA information partially different from DNA information of the donor. Therefore, T cell-derived iPS cells are differentiated into T cells again and used for the purpose of helping the immunity of the donor.

If there is a need to generate iPS cells having the same DNA information as the donor, CD34-positive cells are isolated from blood and reprogrammed to establish iPS cells from CD34-positive cells. CD34-positive cells are undifferentiated blood cells and can also be called blood cell precursor cells. Furthermore, for generating iPS cells having the same DNA information as the donor, monocytes are isolated from blood and reprogrammed to establish iPS cells from monocytes.

### BRIEF DESCRIPTION OF THE DRAWINGS

FIG. 1 is a flowchart showing an example of a method of generating an iPS cell.

FIG. 2 is a diagram showing an example of an iPS cell generation system.

### DETAILED DESCRIPTION

The present inventor focused on the points that the amount of blood collected from the donor is limited, and thus when iPS cells are generated using a single cell type in blood, there is a case where iPS cells cannot be established or cannot be differentiated into target cells after iPS cells are established. For example, if iPS cells are generated using CD34-positive cells, due to the low abundance of CD34-positive cells in blood, there is a case where the sufficient number of cells to establish iPS cells cannot be obtained even if they are proliferated by culture before reprogramming. Furthermore, if iPS cells are generated using monocytes, due to the low iPS cell establishment efficiency thereof, there is a case where iPS cells cannot be established or cannot be differentiated into target cells after iPS cells are established. In such cases, it is necessary to re-establish iPS cells by re-collecting blood from the donor, increasing a burden on the donor and requiring time for establishing iPS cells.

The present inventor newly focused on the above-described problem and tried to solve the novel problem, i.e., working on generating an iPS cell by effectively utilizing the limited amount of blood collected from the donor.

### <1> Method of Generating iPS Cell

In general, according to one embodiment, a method of generating an iPS cell includes:
extracting monocytes from mononuclear cells separated from whole blood to separate the mononuclear cells into the monocytes and non-monocytes;
reprogramming the separated monocytes to generate a monocyte-derived iPS cell; and
culturing the separated non-monocytes in a first culture medium to prepare first raw material cells for iPS cell generation.

The method according to the above-described embodiment may further include:
extracting CD34-positive cells from the first raw material cells to separate the first raw material cells into the CD34-positive cells and non-CD34-positive cells;
reprogramming the separated CD34-positive cells to generate a CD34-positive cell-derived iPS cell; and
culturing the separated non-CD34-positive cells in a second culture medium to prepare second raw material cells for iPS cell generation.

The method according to the above-described embodiment may further include reprogramming the second raw material cells to generate a T cell-derived iPS cell.

### Example of Method of Generating iPS Cell

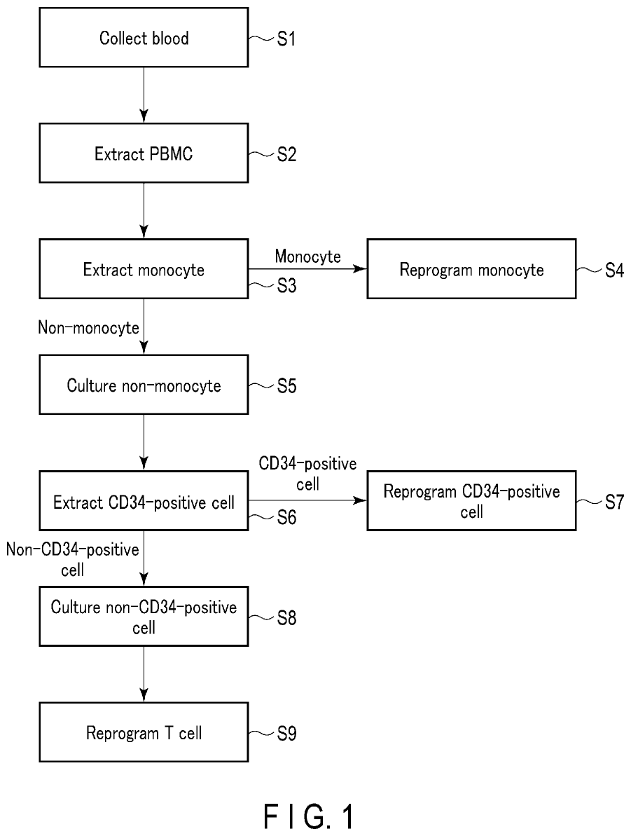

An example of a method of generating an iPS cell is shown in FIG. 1. Hereinafter, an example of a method of generating an iPS cell is explained by following the steps shown in FIG. 1.

### Step of Collecting Blood (S1)

Blood is collected from a donor. Blood collected from the donor is also called whole blood in the description. Blood collected from the donor is generally peripheral blood. An origin of a donor is not limited to a particular animal but is preferably a mammal, more preferably a primate, further more preferably a human.

### Step of Extracting PBMCs (S2)

Peripheral blood mononuclear cells (hereinafter also called PBMCs) are extracted from the blood obtained in the step of collecting blood (S1). Mononuclear cells refer to blood cells having a single circular nucleus, and the mononuclear cells include lymphocytes (T cells, B cells, NK cells), monocytes, and dendritic cells. The mononuclear cells do not include erythrocytes, thrombocytes, or granulocytes. PBMCs refer to mononuclear cells derived from peripheral blood.

PBMCs are extracted by, for example, a density-gradient centrifugation method using a centrifugal tube filled with a density-gradient medium capable of separating PBMCs. As a centrifugal tube, for example, SepMate (Stemcell Technologies) can be used.

### Step of Extracting Monocytes (S3)

Monocytes are extracted from the PBMCs obtained in the step of extracting PBMCs (S2). The PBMCs are thus separated into monocytes and non-monocytes. The monocytes can be extracted by size fractionation, for example, by measuring, with a flow cytometer, the intensity of forward scatter (FSC) reflecting the size of the cells and the intensity of side scatter (SSC) reflecting the internal structure of the cells, but the method is not limited thereto. Monocytes can also be extracted by flow cytometry using a fluorescence-labeled antibody specific for monocytes.

### Step of Reprogramming Monocytes (S4)

The monocytes separated in the step of extracting monocytes (S3) are reprogrammed to generate a monocyte-derived iPS cell. The monocytes can be reprogrammed by introducing reprogramming factors into monocytes using a publicly-known transfection method such as an electroporation method or a lipofection method. The “reprogramming factors” refer to a combination of genes capable of inducing an iPS cell from somatic cells by introducing this gene combination into somatic cells. Various gene combinations are publicly known as reprogramming factors, for example, four factors consisting of Oct3/4, Sox2, Klf4, and c-Myc, and six factors consisting of Oct3/4, Sox2, Klf4, c-Myc, Lin28, and Nanog.

### Step of Culturing Non-Monocytes (S5)

The non-monocytes separated in the step of extracting monocytes (S3) are cultured in a culture medium in parallel with the step of reprogramming monocytes (S4) (i.e., in the same phase as reprogramming monocytes). For the culture medium, it is preferable to use a culture medium for culturing blood cell precursor cells. The culture medium for culturing blood cell precursor cells is commercially available, examples of which include StemSpan SFEM II (Stemcell Technologies). Furthermore, the culture medium may contain a T cell growth factor. The T cell growth factor is a factor that provides growth stimulus on T cells, examples of which include IL-2. The culture can be carried out, for example, for 5 to 7 days.

The culture allows proliferation of CD34-positive cells and T cells contained in the non-monocytes. That is, by this culturing step, it is possible to prepare raw material cells for the subsequent steps of reprogramming CD34-positive cells (S7) and reprogramming T cells (S9).

If CD34-positive cells are proliferated in this culturing step and the number of CD34-positive cells is increased, it is possible to improve iPS cell establishment efficiency in the subsequent step of reprogramming CD34-positive cells (S7). Furthermore, if T cells are proliferated in this culturing step and are in a mitotic phase of the cell cycle, it is possible to improve iPS cell establishment efficiency in the subsequent step of reprogramming T cells (S9).

### Step of Extracting CD34-Positive Cells (S6)

The CD34-positive cells are extracted from the cells obtained in the step of culturing non-monocytes (S5). The non-monocytes are thus separated into CD34-positive cells and non-CD34-positive cells. The CD34-positive cells are blood cell precursor cells. The non-CD34-positive cells predominantly include T cells. The non-CD34-positive cells can also be called CD34-negative cells.

The CD34-positive cells can be extracted by, for example, flow cytometry using a fluorescence-labeled anti-CD34 antibody, but the method is not limited thereto. The CD34-positive cells can be extracted by size fractionation using a flow cytometer, or the CD34-positive cells can also be extracted by analyzing the spectrum of autofluorescence or the intensity at several specific wavelengths (Jpn. Pat. Appln. KOKAI Publication No. 2023-021090).

### Step of Reprogramming CD34-Positive Cells (S7)

The CD34-positive cells separated in the step of extracting CD34-positive cells (S6) are reprogrammed to generate a CD34-positive cell-derived iPS cell. The CD34-positive cells can be reprogrammed by introducing reprogramming factors into CD34-positive cells by a publicly-known transfection method such as an electroporation method or a lipofection method.

### Step of Culturing Non-CD34-Positive Cells (S8)

The non-CD34-positive cells separated in the step of extracting CD34-positive cells (S6) are cultured in a culture medium in parallel with the step of reprogramming CD34-positive cells (S7) (i.e., in the same phase as reprogramming CD34-positive cells). For the culture medium, it is preferable to use a culture medium for culturing T cells. The culture medium for culturing T cells is commercially available, examples of which include X-Vivo 10 (Lonza). Furthermore, it is preferable that the culture medium contain a T cell growth factor. The T cell growth factor is a factor that provides growth stimulus on T cells, examples of which include IL-2, CD3, and CD28 monoclonal antibody. The culture can be carried out, for example, for 5 to 7 days.

The culture allows proliferation of T cells contained in the non-CD34-positive cells. That is, by this culturing step, it is possible to prepare raw material cells for the subsequent step of reprogramming T cells (S9).

If T cells are proliferated in this culturing step and are in a mitotic phase of the cell cycle, it is possible to improve iPS cell establishment efficiency in the subsequent step of reprogramming T cells (S9).

### Step of Reprogramming T Cells (S9)

The cells (including T cells) obtained in the step of culturing non-CD34-positive cells (S8) are reprogrammed to generate a T cell-derived iPS cell. The T cells can be reprogrammed by introducing reprogramming factors into T cells by a publicly-known transfection method such as an electroporation method or a lipofection method.

While the cells used in the step of reprogramming T cells (S9) have undergone two culturing steps (S5 and S8) and it has been, for example, 10 to 14 days since the step of collecting blood (S1), the experiment demonstrated that iPS cells were successfully generated.

### Order of Reprogramming

In the above-described method, culturing non-monocytes (S5) is carried out in parallel with reprogramming monocytes (S4), and after reprogramming monocytes (S4), reprogramming CD34-positive cells (S7) is carried out. Thereafter, culturing non-CD34-positive cells (S8) is carried out in parallel with reprogramming CD34-positive cells (S7), and after reprogramming CD34-positive cells (S7), reprogramming T cells (S9) is carried out. That is, in the above-described method, reprogramming monocytes (S4), reprogramming CD34-positive cells (S7), and reprogramming T cells (S9) are carried out in this order in a series of processes.

The reason why monocytes, CD34-positive cells, and T cells are reprogrammed in this order in the above-described method is explained below. Monocytes are difficult to maintain by culture in vitro, and it is therefore desirable to reprogram monocytes first. CD34-positive cells and T cells can be maintained by culture in vitro, but CD34-positive cells are less abundant in blood and valuable, and it is therefore desirable to reprogram CD34-positive cells promptly after separation. T cells are cultured after separation of non-CD34-positive cells and can improve iPS cell establishment efficiency, and it is therefore desirable to reprogram T cells last.

### First Modification

In the method exemplified above, the step of reprogramming monocytes (S4), the step of reprogramming CD34-positive cells (S7), and the step of reprogramming T cells (S9) are carried out in this order, but in the first modification, the order of the step of reprogramming CD34-positive cells (S7) and the step of reprogramming T cells (S9) may be reversed.

In the first modification, the steps of collecting blood (S1) to extracting CD34-positive cells (S6) are carried out in the same manner as the above-described method. Thereafter, the steps of culturing separated non-CD34-positive cells (S8) and reprogramming T cells (S9) are carried out, and then the step of reprogramming CD34-positive cells (S7) is carried out. That is, in the first modification, iPS cells are generated in the order of monocytes, T cells, and CD34-positive cells.

### Second Modification

While the step of culturing non-monocytes (S5) is carried out in the above-exemplified method, the step of culturing non-monocytes (S5) may be omitted in the second modification.

The second modification can be carried out in the same manner as the above-exemplified method except that the step of culturing non-monocytes (S5) is omitted. That is, in the second modification, the non-monocytes separated in the step of extracting monocytes (S3) are subjected to the step of extracting CD34-positive cells (S6) without being cultured.

### Advantageous Effects

The method according to the embodiment carries out a series of processes for generating iPS cells by separating three types of blood cells (i.e., monocytes, CD34-positive cells, and T cells) from blood of a donor, and reprogramming the three types of blood cells in order. The conventional method, on the other hand, separates a single type of blood cells from blood of a donor and uses only the single type of blood cells for generating iPS cells.

Since the method according to the embodiment carries out a series of processes for generating iPS cells by reprogramming the three types of blood cells in order, the probability of establishing iPS cells is higher than the conventional method, and the risk of not being able to establish iPS cells can be reduced. Therefore, in the method according to the embodiment, a required amount of iPS cells can be secured easily as compared to the conventional method.

Furthermore, the method according to the embodiment can compensate for, with other blood cells, the disadvantages of the respective blood cells that occur when iPS cells are generated by the conventional method, that is, the disadvantage of monocytes that iPS cell establishment efficiency is low, the disadvantage of CD34-positive cells that the number thereof in blood is small, and the disadvantage of T cells that DNA information of T cell-derived iPS cells changes.

Moreover, there is a case where types of differentiated cells into which iPS cells easily differentiate differ depending on types of raw material cells of the iPS cells. Therefore, if iPS cells derived from a plurality of raw material cell types are generated, it is possible to reduce the risk of not being able to differentiate into target cells after establishment of iPS cells (differentiation resistance), and to increase the variation of differentiated cells. That is, if iPS cells derived from a plurality of raw material cell types are generated, it is possible expand the range of iPS cell applications.

As described above, the method according to the embodiment can effectively utilize a limited amount of blood collected from a donor, thereby improving iPS cell establishment efficiency and differentiation efficiency into target cells.

Moreover, in the event that iPS cells cannot be established or cannot be differentiated into target cells, it is necessary to re-collect blood from the donor, re-generate iPS cells, and re-differentiate the iPS cells into target cells. Since the method according to the embodiment can improve iPS cell establishment efficiency and differentiation efficiency into target cells, the possibility of re-collecting blood from a donor can be reduced, and a burden on the donor associated with blood collection can be reduced.

Furthermore, the method according to the embodiment generates iPS cells by reprogramming the three types of blood cells in order and does not carry out the reprogramming processes in parallel at the same time; therefore, the method is excellent in terms of a small risk of mistaking or mixing samples.

### <2> iPS Cell Generation System

A system for carrying out the above-described “Method of Generating iPS Cell” will be described below. In general, according to one embodiment, an iPS cell generation system includes:
a monocyte extraction device for extracting monocytes from mononuclear cells separated from whole blood to separate the mononuclear cells into the monocytes and non-monocytes;
a monocyte reprogramming device for reprogramming the separated monocytes to generate a monocyte-derived iPS cell; and
a non-monocyte culturing device for culturing the separated non-monocytes in a first culture medium to prepare first raw material cells for iPS cell generation.

The system according to the above-described embodiment may further include:
a CD34-positive cell extraction device for extracting CD34-positive cells from the first raw material cells to separate the first raw material cells into the CD34-positive cells and non-CD34-positive cells;
a CD34-positive cell reprogramming device for reprogramming the separated CD34-positive cells to generate a CD34-positive cell-derived iPS cell; and
a non-CD34-positive cell culturing device for culturing the separated non-CD34-positive cells in a second culture medium to prepare second raw material cells for iPS cell generation.

The system according to the above-described embodiment may further include a T cell reprogramming device for reprogramming the second raw material cells to generate a T cell-derived iPS cell.

### Example of iPS Cell Generation System

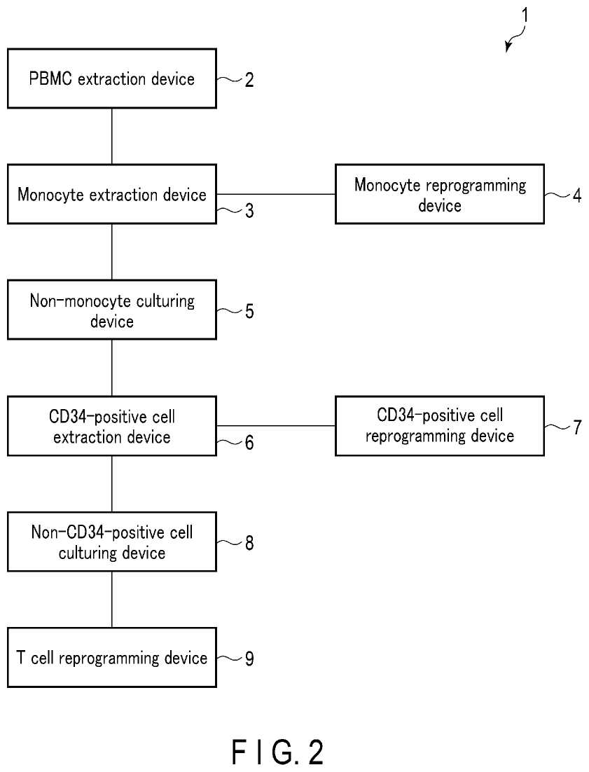

An example of an iPS cell generation system is shown in FIG. 2. As shown in FIG. 2, an iPS cell generation system 1 includes a PBMC extraction device 2, a monocyte extraction device 3, a monocyte reprogramming device 4, a non-monocyte culturing device 5, a CD34-positive cell extraction device 6, a CD34-positive cell reprogramming device 7, a non-CD34-positive cell culturing device 8, and a T cell reprogramming device 9. The cells may be transferred automatically or manually between the devices.

The PBMC extraction device 2 extracts PBMCs from whole blood. As the PBMC extraction device 2, for example, a centrifugal tube filled with a density-gradient medium capable of separating PBMCs and a centrifuge can be used. As the centrifugal tube, for example, SepMate (Stemcell Technologies) can be used.

The monocyte extraction device 3 extracts monocytes from the PBMCs to separate the PBMCs into monocytes and non-monocytes. As the monocyte extraction device 3, for example, a flow cytometer can be used.

The monocyte reprogramming device 4 reprograms the monocytes to generate a monocyte-derived iPS cell. As the monocyte reprogramming device 4, for example, a culture container for containing monocytes and a culture medium, and a first incubator having a culture space inside for culturing the monocytes contained in the culture container under a controlled environment, can be used. The first incubator is configured to control atmospheric conditions (temperature, humidity, gas composition, etc.) of the culture space. For example, the first incubator may include a heater, a humidity controller, a gas supplier, a fan for circulating gas in the culture space, a sensor for measuring the atmospheric conditions in the culture space, and the like.

The non-monocyte culturing device 5 cultures the non-monocytes in a culture medium to prepare first raw material cells for iPS cell generation. As the non-monocyte culturing device 5, for example, a culture container for containing non-monocytes and a culture medium, and a second incubator having a culture space inside for culturing the non-monocytes contained in the culture container under a controlled environment, can be used. As the second incubator, the above-described first incubator may be used.

The CD34-positive cell extraction device 6 extracts CD34-positive cells from the first raw material cells (non-monocytes) to separate the raw material cells into CD34-positive cells and non-CD34-positive cells. As the CD34-positive cell extraction device 6, for example, a flow cytometer can be used. If a flow cytometer is used as the CD34-positive cell extraction device 6, a flow cytometer of the non-monocyte extraction device 3 may be used.

The CD34-positive cell reprogramming device 7 reprograms the CD34-positive cells to generate a CD34-positive cell-derived iPS cell. As the CD34-positive cell reprogramming device 7, for example, a culture container for containing CD34-positive cells and a culture medium, and a third incubator having a culture space inside for culturing the CD34-positive cells contained in the culture container under a controlled environment, can be used. As the third incubator, the above-described first incubator may be used.

The non-CD34-positive cell culturing device 8 cultures the non-CD34-positive cells in a culture medium to prepare second raw material cells for iPS cell generation. As the non-CD34-positive cell culturing device 8, for example, a culture container for containing non-CD34-positive cells and a culture medium, and a fourth incubator having a culture space inside for culturing the non-CD34-positive cells contained in the culture container under a controlled environment, can be used. As the fourth incubator, the above-described first incubator may be used.

The T cell reprogramming device 9 reprograms the second raw material cells (including T cells) to generate a T cell-derived iPS cell. As the T cell reprogramming device 9, for example, a culture container for containing T cells and a culture medium, and a fifth incubator having a culture space inside for culturing the T cells contained in the culture container under a controlled environment, can be used. As the fifth incubator, the above-described first incubator may be used.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A method of generating an iPS cell, the method comprising:
extracting monocytes from mononuclear cells separated from whole blood to separate the mononuclear cells into the monocytes and non-monocytes;
reprogramming the separated monocytes to generate a monocyte-derived iPS cell; and
culturing the separated non-monocytes in a first culture medium to prepare first raw material cells for iPS cell generation.

2. The method according to claim 1, the method further comprising:
extracting CD34-positive cells from the first raw material cells to separate the first raw material cells into the CD34-positive cells and non-CD34-positive cells;
reprogramming the separated CD34-positive cells to generate a CD34-positive cell-derived iPS cell; and
culturing the separated non-CD34-positive cells in a second culture medium to prepare second raw material cells for iPS cell generation.

3. The method according to claim 2, the method further comprising reprogramming the second raw material cells to generate a T cell-derived iPS cell.

4. The method according to claim 1, wherein the first culture medium is a culture medium for culturing blood cell precursor cells.

5. The method according to claim 2, wherein the second culture medium contains a T cell growth factor.

6. The method according to claim 3, wherein the first culture medium is a culture medium for culturing blood cell precursor cells, and the second culture medium contains a T cell growth factor.

7. The method according to claim 1, wherein the culturing of the non-monocytes is carried out in parallel with the reprogramming of the monocytes.

8. The method according to claim 2, wherein the culturing of the non-CD34-positive cells is carried out in parallel with the reprogramming of the CD34-positive cells.

9. The method according to claim 3, wherein the culturing of the non-monocytes is carried out in parallel with the reprogramming of the monocytes, and the culturing of the non-CD34-positive cells is carried out in parallel with the reprogramming of the CD34-positive cells.

10. An iPS cell generation system comprising:
a monocyte extraction device for extracting monocytes from mononuclear cells separated from whole blood to separate the mononuclear cells into the monocytes and non-monocytes;
a monocyte reprogramming device for reprogramming the separated monocytes to generate a monocyte-derived iPS cell; and
a non-monocyte culturing device for culturing the separated non-monocytes in a first culture medium to prepare first raw material cells for iPS cell generation.

11. The iPS cell generation system according to claim 10, the system further comprising:
a CD34-positive cell extraction device for extracting CD34-positive cells from the first raw material cells to separate the first raw material cells into the CD34-positive cells and non-CD34-positive cells;
a CD34-positive cell reprogramming device for reprogramming the separated CD34-positive cells to generate a CD34-positive cell-derived iPS cell; and
a non-CD34-positive cell culturing device for culturing the separated non-CD34-positive cells in a second culture medium to prepare second raw material cells for iPS cell generation.

12. The iPS cell generation system according to claim 11, the system further comprising a T cell reprogramming device for reprogramming the second raw material cells to generate a T cell-derived iPS cell.
